(19) United States
(12) Patent Application Publication
(10) Pub. No.: US 2017/0057889 A1
(43) Pub. Date: Mar. 2, 2017

(54) Method for Producing Hydrocarbons by Oxidative Coupling of Methane with a Heavy Diluent

(71) Applicant: Sabic Global Technologies, B.V., BERGEN OP ZOOM (NL)

(72) Inventors: Vidya Sagar Reddy SARSANI, Pearland, TX (US); David WEST, Bellaire, TX (US); Istvan LENGYEL, Lake Jackson, TX (US)

(21) Appl. No.: 15/241,244

(22) Filed: Aug. 19, 2016

Related U.S. Application Data
(60) Provisional application No. 62/209,561, filed on Aug. 25, 2015.

(51) Int. Cl. C07C 2/84

(52) U.S. Cl. CPC C07C 2/84

(57) ABSTRACT

A method for producing C₂ hydrocarbons comprising (a) introducing a reactant mixture to a reactor comprising a catalyst, wherein the reactant mixture comprises CH₄, O₂ and a heavy diluent, and wherein the reactant mixture is characterized by a bulk CH₄/O₂ molar ratio; (b) allowing the reactant mixture to contact a surface of the catalyst and react via an oxidative coupling of CH₄ (OCM) reaction to form a product mixture, wherein the reactant mixture is characterized by a local CH₄/O₂ molar ratio on the catalyst surface, wherein the local CH₄/O₂ molar ratio is greater than the bulk CH₄/O₂ molar ratio, wherein the product mixture comprises C₂ hydrocarbons, and wherein a selectivity to C₂ hydrocarbons is increased by at least about 1% when compared to a selectivity of an otherwise similar OCM reaction conducted in the absence of the heavy diluent; and (c) recovering the product mixture from the reactor.

### CROSS-REFERENCE TO RELATED APPLICATIONS

The present application is a non-provisional of and claims priority to U.S. Provisional Patent Application No. 62/209,561 filed Aug. 25, 2015 and entitled “Method for Producing Hydrocarbons by Oxidative Coupling of Methane with a Heavy Diluent,” which application is incorporated by reference herein in its entirety.

### TECHNICAL FIELD

The present disclosure relates to methods of producing hydrocarbons, more specifically methods of producing C₂ hydrocarbons by oxidative coupling of methane.

### BACKGROUND

Hydrocarbons, and specifically C₂ hydrocarbons such as ethylene, can be typically used to produce a wide range of products, for example, break-resistant containers and packaging materials. Currently, for industrial scale applications, ethylene is produced by heating natural gas condensates and petroleum distillates, which include ethane and higher hydrocarbons, and the produced ethylene is separated from a product mixture by using gas separation processes.

Ethylene can also be produced by oxidative coupling of the methane (OCM) as represented by Equations (I) and (II):

2CH₄+O₂→C₂H₄+2H₂O ΔH=−67 kcal/mol (I)

2CH₄+½O₂→C₂H₄+H₂O ΔH=−42 kcal/mol (II)

Oxidative conversion of methane to ethylene is exothermic. Excess heat produced from these reactions (Equations (I) and (II)) can push conversion of methane to carbon monoxide and carbon dioxide rather than the desired C₂ hydrocarbon product (e.g., ethylene):

CH₄+1.5O₂→CO+2H₂OΔH=−124 kcal/mol (III)

CH₄+2O₂→CO₂+2H₂OΔH=−192 kcal/mol (IV)

The excess heat from the reactions in Equations (III) and (IV) further exasperate this situation, thereby substantially reducing the selectivity of ethylene production when compared with carbon monoxide and carbon dioxide production.

Additionally, while the overall OCM is exothermic, catalysts are used to overcome the endothermic nature of the C—H bond breakage. The endothermic nature of the bond breakage is due to the chemical stability of methane, which is a chemically stable molecule due to the presence of its four strong tetrahedral C—H bonds (435 kJ/mol). When catalysts are used in the OCM, the exothermic reaction can lead to a large increase in catalyst bed temperature and uncontrolled heat excursions that can lead to catalyst deactivation and a further decrease in ethylene selectivity. Furthermore, the produced ethylene is highly reactive and can form unwanted and thermodynamically favored oxidation products.

There have been attempts to control the exothermic reaction of the OCM by using alternating layers of selective OCM catalysts; through the use of fluidized bed reactors; and/or by using steam as a diluent. However, these solutions are costly and inefficient. For example, a large amount of water (steam) is required to absorb the heat of the reaction. Thus, there is an ongoing need for the development of OCM processes.

### BRIEF SUMMARY

Disclosed herein is a method for producing C₂ hydrocarbons comprising (a) introducing a reactant mixture to a reactor comprising a catalyst, wherein the reactant mixture comprises methane (CH₄), oxygen (O₂) and a heavy diluent, and wherein the reactant mixture is characterized by a bulk CH₄/O₂ molar ratio, (b) allowing at least a portion of the reactant mixture to contact a surface of the catalyst and react via an oxidative coupling of CH₄ reaction to form a product mixture, wherein the reactant mixture is characterized by a local CH₄/O₂ molar ratio on the catalyst surface, wherein the local CH₄/O₂ molar ratio is greater than the bulk CH₄/O₂ molar ratio, wherein the product mixture comprises C₂ hydrocarbons, and wherein a selectivity to C₂ hydrocarbons is increased by equal to or greater than about 1% when compared to a selectivity of an otherwise similar oxidative coupling of CH₄ reaction conducted with a similar bulk CH₄/O₂ molar ratio in the absence of the heavy diluent, and (c) recovering at least a portion of the product mixture from the reactor.

Also disclosed herein is a method for producing ethylene comprising (a) introducing a reactant mixture to a reactor comprising a catalyst, wherein the reactant mixture comprises methane (CH₄), oxygen (O₂) and carbon dioxide (CO₂), and wherein the reactant mixture is characterized by a bulk CH₄/O₂ molar ratio of from about 4:1 to about 8:1, (b) allowing at least a portion of the reactant mixture to contact a surface of the catalyst and react via an oxidative coupling of CH₄ reaction to form a product mixture, wherein the reactant mixture is characterized by a local CH₄/O₂ molar ratio on the catalyst surface, wherein the local CH₄/O₂ molar ratio is greater than the bulk CH₄/O₂ molar ratio, wherein the product mixture comprises C₂ hydrocarbons, and wherein a selectivity to C₂ hydrocarbons is increased by equal to or greater than about 5% when compared to a selectivity of an otherwise similar oxidative coupling of CH₄ reaction conducted with a similar reactant mixture lacking CO₂, (c) recovering at least a portion of the product mixture from the reactor, (d) separating at least a portion of C₂₊ hydrocarbons from the product mixture to yield recovered C₂₊ hydrocarbons, and (e) using at least a portion of the recovered C₂₊ hydrocarbons to produce ethylene.

### DETAILED DESCRIPTION

Disclosed herein are methods for producing C₂ hydrocarbons comprising (a) introducing a reactant mixture to a reactor comprising a catalyst, wherein the reactant mixture comprises methane (CH₄), oxygen (O₂) and a heavy diluent, and wherein the reactant mixture is characterized by a bulk CH₄/O₂ molar ratio; (b) allowing at least a portion of the reactant mixture to contact a surface of the catalyst and react via an oxidative coupling of CH₄ reaction to form a product mixture, wherein the reactant mixture is characterized by a local CH₄/O₂ molar ratio on the catalyst surface, wherein the local CH₄/O₂ molar ratio is greater than the bulk CH₄/O₂ molar ratio, wherein the product mixture comprises C₂ hydrocarbons, and wherein a selectivity to C₂ hydrocarbons is increased by equal to or greater than about 1% when compared to a selectivity of an otherwise similar oxidative coupling of CH₄ reaction conducted with a similar bulk CH₄/O₂ molar ratio in the absence of the heavy diluent; and (c) recovering at least a portion of the product mixture from the reactor. In such embodiment, the heavy diluent comprises carbon dioxide (CO₂), silicon tetrafluoride (SiF₄), carbon tetrafluoride (CF₄), a heavy inert gas, argon (Ar), krypton (Kr), and the like, or combinations thereof. In such embodiment, the C₂ hydrocarbons comprise ethylene.

Other than in the operating examples or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an embodiment,” “another embodiment,” “other embodiments,” “some embodiments,” and so forth, means that a particular element (e.g., feature, structure, property, and/or characteristic) described in connection with the embodiment is included in at least an embodiment described herein, and may or may not be present in other embodiments. In addition, it is to be understood that the described element(s) can be combined in any suitable manner in the various embodiments.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art.

Compounds are described herein using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through the carbon of the carbonyl group.

In an embodiment, a method for producing C₂ hydrocarbons can comprise introducing a reactant mixture to a reactor comprising a catalyst, wherein the reactant mixture comprises methane (CH₄), oxygen (O₂) and a heavy diluent; and allowing at least a portion of the reactant mixture to contact a surface of the catalyst and react via an oxidative coupling of CH₄ (OCM) reaction to form a product mixture.

The OCM has been the target of intense scientific and commercial interest for more than thirty years due to the tremendous potential of such technology to reduce costs, energy, and environmental emissions in the production of ethylene (C₂H₄). As an overall reaction, in the OCM, CH₄ and O₂ react exothermically over a catalyst to form C₂H₄, water (H₂O) and heat.

Generally, in the OCM, CH₄ is first oxidatively converted into ethane (C₂H₆), and then into C₂H₄. CH₄ is activated heterogeneously on a catalyst surface, forming methyl free radicals (e.g., CH₃—), which then couple in a gas phase to form C₂H₆. C₂H₆ subsequently undergoes dehydrogenation to form C₂H₄. An overall yield of desired C₂ hydrocarbons is reduced by non-selective reactions of methyl radicals with the catalyst surface and/or oxygen in the gas phase, which produce (undesirable) carbon monoxide and carbon dioxide. Some of the best reported OCM outcomes encompass a ˜20% conversion of methane and −80% selectivity to desired C₂ hydrocarbons.

In an embodiment, the reactant mixture can comprise a hydrocarbon or mixtures of hydrocarbons, oxygen and a heavy diluent. In some embodiments, the hydrocarbon or mixtures of hydrocarbons can comprise natural gas (e.g., CH₄), liquefied petroleum gas comprising C₂-C₅ hydrocarbons, C₆₊ heavy hydrocarbons (e.g., C₆ to C₂₄ hydrocarbons such as diesel fuel, jet fuel, gasoline, tars, kerosene, etc.), oxygenated hydrocarbons, biodiesel, alcohols, dimethyl ether, and the like, or combinations thereof. In an embodiment, the reactant mixture can comprise CH₄, O₂ and a heavy diluent.

In an embodiment, the O₂ used in the reaction mixture can be oxygen gas (which may be obtained via a membrane separation process), technical oxygen (which may contain some air), air, oxygen enriched air, and the like, or combinations thereof.

In an embodiment, the reactant mixture can be a gaseous mixture. In an embodiment, the reactant mixture can be characterized by a bulk CH₄/O₂ molar ratio, e.g., a molar ratio of the CH₄ and the O₂ as they enter a reactor, and prior to contacting a catalyst surface and/or engaging in any chemical reaction.

In an embodiment, the bulk CH₄/O₂ molar ratio can be from about 1:1 to about 20:1, alternatively from about 1:1 to about 16:1, alternatively from about 2:1 to about 12:1, alternatively from about 3:1 to about 9:1, alternatively from about 4:1 to about 8:1, or alternatively from about 4:1 to about 6:1. As will be appreciated by one of skill in the art, and with the help of this disclosure, the greater the bulk CH₄/O₂ molar ratio, the greater a selectivity to desired C₂ hydrocarbons, and the lower the CH₄ conversion.

In an embodiment, the reactant mixture can comprise a heavy diluent comprising carbon dioxide (CO₂), silicon tetrafluoride (SiF₄), carbon tetrafluoride (CF₄), a heavy inert gas, argon (Ar), krypton (Kr), and the like, or combinations thereof. In an embodiment, the heavy diluent comprises CO₂. For purposes of the disclosure herein, a heavy inert gas refers to an inert gas (e.g., a gas that does not participate in the OCM reaction) characterized by a molecular weight of equal to or greater than about 35 g/mol.

In an embodiment, the heavy diluent can form a heavy diluent-rich thin layer at the surface of the catalyst. The heavy diluent is inert with respect to the OCM reaction, e.g., the heavy diluent does not participate in the OCM reaction. For purposes of the disclosure herein, a heavy diluent-rich thin layer refers to a thin layer that comprises a heavy diluent in an amount that is increased by at least 1 mol %, alternatively by at least 2 mol %, alternatively by at least 3 mol %, alternatively by at least 4 mol %, or alternatively by at least 5 mol %, when compared to an amount of heavy diluent present in a bulk of the reactant mixture (as opposed to a reactant mixture at a catalyst surface). The heavy diluent-rich thin layer can further comprise components other than the heavy diluent, such as for example an optional light diluent (e.g., water, light inert gases, nitrogen, etc.), OCM reaction products, C₂₊ hydrocarbons, CO, H₂, and the like, or combinations thereof.

In an embodiment, the heavy diluent can be characterized by a molecular weight of from about 35 g/mol to about 125 g/mol, alternatively from about 40 g/mol to about 110 g/mol, or alternatively from about 44 g/mol to about 105 g/mol. Without wishing to be limited by theory, the molecular weight of the heavy diluent is greater than the molecular weight of either CH₄ or O₂.

In an embodiment, the CH₄ diffuses faster than O₂ through the heavy diluent-rich thin layer, at a given temperature. According to Graham's law of effusion (e.g., a process in which a gas escapes through a small hole), the relative rates of effusion of two gases at the same temperature and pressure are given by the inverse ratio of the square roots of the molecular weights of the respective gas particles. Graham's law also approximates very well the diffusion of gasses, and as such it indicates that in a given medium (e.g., heavy diluent, CO₂, etc.) gases of lower molecular weight (e.g., CH₄) diffuse faster than gases of higher molecular weight (e.g., O₂), at a given temperature (and pressure).

In an embodiment, the reactant mixture can be characterized by a diffusivity ratio of CH₄/O₂ in a gas mixture comprising the heavy diluent (e.g., reactant mixture) that is increased by equal to or greater than about 5%, alternatively by equal to or greater than about 10%, or alternatively by equal to or greater than about 15%, when compared to a diffusivity ratio of CH₄/O₂ in an otherwise similar gas mixture lacking the heavy diluent, at a given temperature. Without wishing to be limited by theory, when diffusing through a medium of a higher molecular weight (e.g., a gas mixture such as the reactant mixture comprising the heavy diluent such as CO₂, as opposed to H₂O), a rate of diffusion of a given gas will be decreased; and the higher the molecular weight of the diffusing gas, the higher the magnitude of the decrease in the rate of diffusion.

In an embodiment, the heavy diluent can further comprise water, light inert gases, nitrogen, and the like, or combinations thereof. For purposes of the disclosure herein, a light inert gas refers to an inert gas (e.g., a gas that does not participate in the OCM reaction) characterized by a molecular weight of less than about 35 g/mol.

In an embodiment, the reactant mixture can be characterized by a local CH₄/O₂ molar ratio on the catalyst surface, wherein the local CH₄/O₂ molar ratio is greater than the bulk CH₄/O₂ molar ratio. Without wishing to be limited by theory, as the CH₄ diffuses faster than O₂ through the heavy diluent-rich thin layer, there will be relatively more CH₄ reaching the surface of the catalyst as compared to an otherwise similar reaction mixture lacking a heavy diluent, thereby leading to a local CH₄/O₂ molar ratio on the catalyst surface that is greater than the bulk CH₄/O₂ molar ratio fed to the reactor.

In some embodiments, the heavy diluent can physically interact with the catalyst (e.g., a portion of the heavy diluent can be adsorbed on the catalyst surface) thereby decreasing catalyst activity. Without wishing to be limited by theory, when the heavy diluent is adsorbed onto the catalyst surface, fewer catalyst active sites are available for the OCM, and consequently the overall rate of the OCM is slower (as opposed to no heavy diluent adsorbed onto the catalyst surface), thereby allowing more time for removing the heat produced by the exothermic OCM reaction.

In an embodiment, the heavy diluent can provide for heat control of the OCM reaction, e.g., the heavy diluent can act as a heat sink. Generally, an inert compound (e.g., a heavy diluent) can absorb some of the heat produced in the exothermic OCM reaction, without degrading or participating in any reaction (OCM or other reaction), thereby providing for controlling a temperature inside the reactor

In an embodiment, the heavy diluent can be characterized by a thermal stability of equal to or less than about 1,200° C., alternatively equal to or less than about 1,100° C., or alternatively equal to or less than about 1,000° C. Generally, the thermal stability of a compound (e.g., heavy diluent) refers to a temperature up to which the compound is stable, e.g., does not decompose or degrade.

In an embodiment, the heavy diluent can be present in the reactant mixture in an amount of from about 10 mol % to about 80 mol %, alternatively from about 15 mol % to about 75 mol %, or alternatively from about 20 mol % to about 70 mol %.

In an embodiment, a method for producing C₂ hydrocarbons can comprise introducing the reactant mixture to a reactor comprising a catalyst. In such embodiment, the reactor can comprise an adiabatic reactor, an autothermal reactor, a tubular reactor, a cooled tubular reactor, a continuous flow reactor, a fixed bed reactor, a fluidized bed reactor, and the like, or combinations thereof.

In an embodiment, the reaction mixture can be introduced to the reactor at a temperature of from about 150° C. to about 300° C., alternatively from about 175° C. to about 250° C., or alternatively from about 200° C. to about 225° C. As will be appreciated by one of skill in the art, and with the help of this disclosure, while the OCM reaction is exothermic, heat input is necessary for promoting the formation of methyl radicals from CH₄, as the C—H bonds of CH₄ are very stable, and the formation of methyl radicals from CH₄ is endothermic. In an embodiment, the reaction mixture can be introduced to the reactor at a temperature effective to promote an OCM reaction.

In an embodiment, the reactor can be characterized by a temperature of from about 400° C. to about 1,200° C., alternatively from about 500° C. to about 1,100° C., or alternatively from about 600° C. to about 1,000° C. As will be appreciated by one of skill in the art, and with the help of this disclosure, different catalysts have different deactivation temperatures, and as such the reactor temperature can vary based on the type of catalyst used.

In an embodiment, the reactor can be characterized by a pressure of from about ambient pressure (e.g., atmospheric pressure) to about 500 psig, alternatively from about ambient pressure to about 200 psig, or alternatively from about ambient pressure to about 100 psig. In an embodiment, the method for producing C₂ hydrocarbons as disclosed herein can be carried out at ambient pressure.

In an embodiment, the reactor can be characterized by a residence time of from about 1 millisecond (ms) to about 2 seconds (s), alternatively from about 10 ms to about 1 s, or alternatively from about 15 ms to about 500 ms. Generally, the residence time of a reactor refers to the average amount of time that a compound (e.g., a molecule of that compound) spends in that particular reactor.

In an embodiment, the reactor can be characterized by a weight hourly space velocity of from about 1,000 h⁻¹ to about 1,000,000 h⁻¹, alternatively from about 5,000 h⁻¹ to about 100,000 h⁻¹, or alternatively from about 10,000 h⁻¹ to about 25,000 h⁻¹. Generally, the weight hourly space velocity refers to a mass of reagents fed per hour divided by a mass of catalyst used in a particular reactor.

In an embodiment, the reactor can comprise a catalyst, wherein the catalyst catalyzes the OCM reaction. In such embodiment, the catalyst can comprise basic oxides; mixtures of basic oxides; redox elements; redox elements with basic properties; mixtures of redox elements with basic properties; mixtures of redox elements with basic properties promoted with alkali and/or alkaline earth metals; rare earth metal oxides; mixtures of rare earth metal oxides; mixtures of rare earth metal oxides promoted by alkali and/or alkaline earth metals; manganese; manganese compounds; lanthanum; lanthanum compounds; sodium; sodium compounds; cesium; cesium compounds; calcium; calcium compounds; and the like; or combinations thereof.

In an embodiment, the catalysts suitable for use in the present disclosure can be supported catalysts and/or unsupported catalysts. In some embodiments, the supported catalyst can comprise a support, wherein the support can be catalytically active (e.g., the support can catalyze an OCM reaction). In other embodiments, the supported catalyst can comprise a support, wherein the support can be catalytically inactive (e.g., the support cannot catalyze an OCM reaction). In yet other embodiments, the supported catalyst can comprise a catalytically active support and a catalytically inactive support. Nonlimiting examples of a catalyst support suitable for use in the present disclosure include MgO, Al₂O₃, SiO₂, and the like, or combinations thereof. As will be appreciated by one of skill in the art, and with the help of this disclosure, the support can be purchased or can be prepared by using any suitable methodology, such as for example precipitation/co-precipitation, sol-gel techniques, templates/surface derivatized metal oxides synthesis, solid-state synthesis of mixed metal oxides, microemulsion techniques, solvothermal techniques, sonochemical techniques, combustion synthesis, etc.

In an embodiment, the catalyst can comprise one or more metals (e.g., catalytic metals), one or more metal compounds (e.g., compounds of catalytic metals), and the like, or combinations thereof. Nonlimiting examples of catalytic metals suitable for use in the present disclosure include Li, Na, Ca, Cs, Mg, La, Ce, W, Mn, and the like, or combinations thereof. Nonlimiting examples of catalysts suitable for use in the present disclosure include La on a MgO support, Na, Mn, and La₂O₃ on an alumina support, Na and Mn oxides on a silicon dioxide support, Na₂WO₄ and Mn on a silicon dioxide support, and the like, or combinations thereof.

In an embodiment, a catalyst that can promote an OCM reaction to produce ethylene can comprise Li₂O, Na₂O, Cs₂O, MgO, WO₃, Mn₃O₄, and the like, or combinations thereof. In some embodiments, the catalyst can comprise a catalyst mixture, such as for example a catalyst mixture comprising a first supported catalyst comprising Ce and La, and a second supported catalyst comprising Mn, W, and Na.

Nonlimiting examples of catalysts suitable for use in the present disclosure include CaO, MgO, BaO, CaO—MgO, CaO—BaO, Li/MgO, MnO₂, W₂O₃, SnO₂, MnO₂—W₂O₃, MnO₂—W₂O₃—Na₂O, MnO₂—W₂O₃—Li₂O, La₂O₃, SrO/La₂O₃, CeO₂, Ce₂O₃, La/MgO, La₂O₃—CeO₂, La₂O₃—CeO₂—Na₂O, La₂O₃—CeO₂—CaO, Na—Mn—La₂O₃/Al₂O₃, Na—Mn—O/SiO₂, Na₂WO₄—Mn/SiO₂, Na₂WO₄—Mn—O/SiO₂, and the like, or combinations thereof.

In an embodiment, the method for producing C₂ hydrocarbons as disclosed herein further excludes CO₂ reforming of CH₄. Generally, CO₂ reforming of CH₄ refers to an endothermic process by which CO₂ and CH₄ are catalytically converted to synthesis gas (e.g., CO and H₂). As will be appreciated by one of skill in the art, and with the help of this disclosure, when used in the reaction mixture, CO₂ is a heavy diluent, and is not intended to participate in any chemical reaction, such as CO₂ reforming of CH₄.

In an embodiment, the catalyst does not catalyze CO₂ reforming of CH₄. In an embodiment, the catalyst excludes nickel, a noble metal, rhodium, ruthenium, platinum, palladium, and the like, or combinations thereof.

In an embodiment, a method for producing C₂ hydrocarbons can comprise allowing at least a portion of the reactant mixture to contact a surface of the catalyst and react via an OCM reaction to form a product mixture, wherein the product mixture comprises C₂ hydrocarbons, and wherein a selectivity to C₂ hydrocarbons (e.g., C₂ selectivity) is increased by equal to or greater than about 1%, alternatively equal to or greater than about 2%, alternatively equal to or greater than about 3%, alternatively equal to or greater than about 4%, alternatively equal to or greater than about 5%, alternatively equal to or greater than about 6%, alternatively equal to or greater than about 7%, alternatively equal to or greater than about 8%, alternatively equal to or greater than about 9%, alternatively equal to or greater than about 10%, alternatively equal to or greater than about 11%, alternatively equal to or greater than about 12%, alternatively equal to or greater than about 13%, alternatively equal to or greater than about 14%, or alternatively equal to or greater than about 15%, when compared to a selectivity of an otherwise similar OCM reaction conducted with a similar bulk CH₄/O₂ molar ratio in the absence of the heavy diluent. Generally, a selectivity to a desired product or products refers to how much desired product was formed divided by the total products formed, both desired and undesired. For purposes of the disclosure herein, the selectivity to a desired product is a % selectivity based on moles converted into the desired product. Further, for purposes of the disclosure herein, a Cₓ selectivity (e.g., C₂ selectivity, C₂₊ selectivity, etc.) can be calculated by dividing a number of moles of carbon (C) from CH₄ that were converted into the desired product (e.g., C_{C2H4}, C_{C2H6}, etc.) by the total number of moles of C from CH₄ that were converted (e.g., C_{C2H4}, C_{C2H6}, C_{C2H2}, C_{C3H6}, C_{C3H8}, C_{C4s}, C_{CO2}, C_{CO}, etc.). C_{C2H4}=number of moles of C from CH₄ that were converted into C₂H₄; C_{C2H6}=number of moles of C from CH₄ that were converted into C₂H₆; C_{C2H2}=number of moles of C from CH₄ that were converted into C₂H₂; C_{C3H6}=number of moles of C from CH₄ that were converted into C₃H₆; C_{C3H8}=number of moles of C from CH₄ that were converted into C₃H₈, C_{C4s}=number of moles of C from CH₄ that were converted into C₄ hydrocarbons (C₄s); C_{CO2}=number of moles of C from CH₄ that were converted into CO₂; C_{CO}=number of moles of C from CH₄ that were converted into CO; etc.

In an embodiment, the product mixture comprises C₂₊ hydrocarbons, wherein the C₂₊ hydrocarbons comprise C₂ hydrocarbons and C₃ hydrocarbons. In an embodiment, the C₂ hydrocarbons can comprise C₂H₄ and C₂H₆. In an embodiment, the C₂ hydrocarbons can further comprise acetylene (C₂H₂). In an embodiment, the C₃ hydrocarbons can comprise propylene (C₃H₆) and propane (C₃H₈). In an embodiment, the C₂₊ hydrocarbons can further comprise C₄ hydrocarbons (C₄s), such as for example butane, iso-butane, n-butane, butylene, etc.

In an embodiment, a C₂₊ selectivity (e.g., selectivity to C₂₊ hydrocarbons) refers to how much C₂H₄, C₃H₆, C₂H₂, C₂H₆, C₃H₈, and C₄s were formed divided by the total products formed, including C₂H₄, C₃H₆, C₂H₂, C₂H₆, C₃H₈, C₄s, CO₂ and CO. For example, the C₂₊ selectivity can be calculated by using equation (1):

$${C}_{2 +} selectivity = \frac{2 {C}_{{C}_{2} {H}_{4}} + 2 {C}_{{C}_{2} {H}_{6}} + 2 {C}_{{C}_{2} {H}_{2}} + 3 {C}_{{C}_{3} {H}_{6}} + 3 {C}_{{C}_{3} {H}_{5}} + 4 {C}_{{C}_{4 s}}}{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4 s}} + {C}_{{CO}_{2}} + {C}_{CO}\end{matrix}} \times 100 \% \qquad ( 1 )$$

As will be appreciated by one of skill in the art, if a specific product and/or hydrocarbon product is not produced in a certain OCM reaction/process, then the corresponding C_{Cx} is 0, and the term is simply removed from selectivity calculations.

In an embodiment, the C₂₊ selectivity (e.g., selectivity to C₂₊ hydrocarbons) can be from about 55% to about 95%, alternatively from about 60% to about 90%, or alternatively from about 65% to about 85%.

In an embodiment, the C₂ selectivity refers to how much C₂H₄ and C₂H₆ were formed divided by the total products formed, including C₂H₄, C₃H₆, C₂H₂, C₂H₆, C₃H₈, C₄s, CO₂ and CO. For example, the C₂ selectivity can be calculated by using equation (2):

$${C}_{2} selectivity = \frac{2 {C}_{{C}_{2} {H}_{4}} + 2 {C}_{{C}_{2} {H}_{6}} + 2 {C}_{{C}_{2} {H}_{2}}}{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 4 {C}_{{C}_{4 s}} + {C}_{{CO}_{2}} + {C}_{CO}\end{matrix}} \times 100 \% \qquad ( 2 )$$

In an embodiment, the C₂ selectivity (e.g., selectivity to C₂ hydrocarbons) can be from about 50% to about 90%, alternatively from about 55% to about 80%, or alternatively from about 60% to about 75%.

In an embodiment, a selectivity to ethylene (C₂₌ selectivity) can be from about 30% to about 50%, alternatively from about 32.5% to about 47.5%, or alternatively from about 35% to about 45%. For example, the selectivity to ethylene can be calculated by using equation (3):

$${C}_{2 =} selectivity = \frac{2 {C}_{{C}_{2} {H}_{4}}}{\begin{matrix}2 {C}_{{C}_{2} {H}_{4}} + 2 {C}_{{C}_{2} {H}_{6}} + 2 {C}_{{C}_{2} {H}_{2}} + 3 {C}_{{C}_{3} {H}_{6}} + \\ 3 {C}_{{C}_{3} {H}_{8}} + 4 {C}_{{C}_{4 s}} + {C}_{{CO}_{2}} + {C}_{CO}\end{matrix}} \times 100 \% \qquad ( 3 )$$

In an embodiment, a methane conversion can be from about 5% to about 50%, alternatively from about 10% to about 45%, or alternatively from about 15% to about 40%. Generally, a conversion of a reagent or reactant refers to the percentage (usually mol %) of reagent that reacted to both undesired and desired products, based on the total amount (e.g., moles) of reagent present before any reaction took place. For purposes of the disclosure herein, the conversion of a reagent is a % conversion based on moles converted. For example, the methane conversion can be calculated by using equation (4):

$${CH}_{4} conversion = \frac{{C}_{{CH}_{4}}^{in} - {C}_{{CH}_{4}}^{out}}{{C}_{{CH}_{4}}^{in}} \times 100 \% \qquad ( 4 )$$

wherein C_{CH}_{₄}ⁱⁿ=number of moles of C from CH₄ that entered the reactor as part of the reactant mixture; and C_{CH}_{₄}^{out}=number of moles of C from CH₄ that was recovered from the reactor as part of the product mixture.

In an embodiment, a sum of CH₄ conversion plus the selectivity to C₂₊ hydrocarbons can be equal to or greater than about 100%, alternatively equal to or greater than about 105%, or alternatively equal to or greater than about 110%. As the CH₄ conversion decreases with increasing the bulk CH₄/O₂ molar ratio, using a lower bulk CH₄/O₂ molar ratio can still provide for a higher methane conversion, owing to an increased local CH₄/O₂ molar ratio on the catalyst surface. Further, a desired selectivity to C₂ hydrocarbons can be obtained when a heavy diluent is used, as the heavy diluent provides for an increased local CH₄/O₂ molar ratio on the catalyst surface, thereby providing for an increased selectivity to desired C₂ hydrocarbons.

In an embodiment, equal to or greater than about 5 mol %, alternatively equal to or greater than about 10 mol %, or alternatively equal to or greater than about 15 mol % of the reactant mixture can be converted to ethylene.

In an embodiment, equal to or greater than about 10 mol %, alternatively equal to or greater than about 15 mol %, or alternatively equal to or greater than about 20 mol % of the reactant mixture can be converted to C₂ hydrocarbons.

In an embodiment, equal to or greater than about 12 mol %, alternatively equal to or greater than about 17 mol %, or alternatively equal to or greater than about 22 mol % of the reactant mixture can be converted to C₂₊ hydrocarbons.

In an embodiment, a method for producing C₂ hydrocarbons can comprise recovering at least a portion of the product mixture from the reactor. In an embodiment, the product mixture can comprise at least a portion of the heavy diluent and unreacted methane. When water (e.g., steam) is used as part of the reaction mixture, the water can be separated from the product mixture prior to separating any of the other product mixture components. For example, by cooling down the product mixture to a temperature where the water condenses (e.g., below 100° C. at ambient pressure), the water can be removed from the product mixture, by using a flash chamber for example.

In an embodiment, at least a portion of the heavy diluent can be separated from the product mixture to yield a recovered heavy diluent. The heavy diluent can be separated from the product mixture by using any suitable commercially available separation techniques. In an embodiment, at least a portion of the heavy diluent can be separated from the product mixture by distillation. In an embodiment, at least a portion of the recovered heavy diluent can be recycled to the reactant mixture.

In embodiments wherein the heavy diluent comprises CO₂, the heavy diluent can be separated from the product mixture by amine absorption, followed by a caustic wash.

In an embodiment, at least a portion of the C₂₊ hydrocarbons can be separated from the product mixture to yield recovered C₂₊ hydrocarbons. The C₂₊ hydrocarbons can be separated from the product mixture by using any suitable separation technique. In an embodiment, at least a portion of the C₂₊ hydrocarbons can be separated from the product mixture by distillation (e.g., cryogenic distillation).

In an embodiment, at least a portion of the recovered C₂₊ hydrocarbons can be used for ethylene production. In some embodiments, at least a portion of ethylene can be separated from the product mixture by using any suitable separation technique (e.g., distillation). In other embodiments, at least a portion of the recovered C₂₊ hydrocarbons can be converted to ethylene, for example by steam cracking.

In an embodiment, at least a portion of the unreacted methane can be separated from the product mixture to yield recovered methane. Methane can be separated from the product mixture by using any suitable separation technique, such as for example distillation (e.g., cryogenic distillation). In an embodiment, at least a portion of the recovered methane can be recycled to the reactant mixture.

In an embodiment, the product mixture can further comprise synthesis gas (e.g., CO and H₂). Synthesis gas, also known as syngas, is generally a gas mixture consisting primarily of CO and H₂, and sometimes CO₂. Synthesis gas can be used for producing methanol; for producing olefins; for producing ammonia and fertilizers; in the steel industry; as a fuel source (e.g., for electricity generation); etc.

In an embodiment, at least a portion of the unreacted methane and at least a portion of the synthesis gas can be separated from the product mixture to yield a fuel gas mixture, wherein the fuel gas mixture can comprise CO, H₂, and CH₄. In an embodiment, at least a portion of the fuel gas mixture can be used as a source of fuel for generating energy.

In an embodiment, a method for producing ethylene can comprise (a) introducing a reactant mixture to a reactor comprising a catalyst, wherein the reactant mixture comprises methane (CH₄), oxygen (O₂) and carbon dioxide (CO₂) and wherein the reactant mixture can be characterized by a bulk CH₄/O₂ molar ratio of from about 4:1 to about 8:1; (b) allowing at least a portion of the reactant mixture to contact a surface of the catalyst and react via an oxidative coupling of CH₄ reaction to form a product mixture, wherein the reactant mixture can be characterized by a local CH₄/O₂ molar ratio on the catalyst surface, wherein the local CH₄/O₂ molar ratio can be greater than the bulk CH₄/O₂ molar ratio, wherein the product mixture can comprise C₂ hydrocarbons, and wherein a selectivity to C₂ hydrocarbons can be increased by equal to or greater than about 5% when compared to a selectivity of an otherwise similar oxidative coupling of CH₄ reaction conducted with a similar reactant mixture lacking CO₂; (c) recovering at least a portion of the product mixture from the reactor; (d) separating at least a portion of C₂₊ hydrocarbons from the product mixture to yield recovered C₂₊ hydrocarbons; and (e) using at least a portion of the recovered C₂₊ hydrocarbons to produce ethylene. In such embodiment, (e) using at least a portion of the recovered C₂₊ hydrocarbons to produce ethylene can comprise (i) separating ethylene from the recovered C₂₊ hydrocarbons to yield recovered ethylene by distillation and/or (ii) converting the recovered C₂₊ hydrocarbons to ethylene by steam cracking. In an embodiment, the method for producing ethylene further excludes CO₂ reforming of CH₄.

In an embodiment, a method for producing C₂ hydrocarbons (e.g., ethylene) as disclosed herein can advantageously display improvements in one or more method characteristics when compared to an otherwise similar method lacking using a reactant mixture comprising a heavy diluent. In an embodiment, the method for producing C₂ hydrocarbons (e.g., ethylene) as disclosed herein can advantageously display an enhanced C₂₊ selectivity when compared to an otherwise similar method of producing C₂₊ hydrocarbons lacking a heavy diluent in the reactant mixture. Such increase in C₂₊ selectivity can advantageously lead to a sum of methane conversion plus C₂₊ selectivity of greater than about 100%.

In an embodiment, a method for producing C₂ hydrocarbons (e.g., ethylene) as disclosed herein can advantageously provide for better reaction temperature control when compared to an otherwise similar method lacking using a reactant mixture comprising a heavy diluent, owing to the heavy diluent acting as a heat sink. Additional advantages of the methods for the production of C₂ hydrocarbons (e.g., ethylene) as disclosed herein can be apparent to one of skill in the art viewing this disclosure.

### EXAMPLES

The subject matter having been generally described, the following examples are given as particular embodiments of the disclosure and to demonstrate the practice and advantages thereof. It is understood that the examples are given by way of illustration and are not intended to limit the specification of the claims to follow in any manner.

### Example 1

Oxidative coupling of methane (OCM) reactions were conducted in the absence of a heavy diluent as follows. The mixture of methane and oxygen along with an internal standard, an inert gas (e.g., neon) were fed to the small quartz reactor with an internal diameter (I.D.) of 4 mm, which was located in a traditional clamshell furnace. A catalyst loading was 100 mg, and total flow rates of the gases corresponded to a desired residence time. The residence times ranged from about 25 ms to about 130 ms for the data displayed in Table 1. The reactor was first heated to a desired temperature under an inert gas flow and then a desired gas mixture was fed to the reactor.

Selectivities and conversions were calculated as outlined in equations (1)-(4), and the data are di splayed in Table 1.

TABLE 1

| | | | | |
|---|---|---|---|---|
| Feed CH₄/O₂ molar ratio | 12 | 7.5 | 4 | 2.5 |
| % CH₄ Conversion | 14.0 | 18.9 | 30.6 | 44.0 |
| % O₂ Conversion | 100.0 | 98.7 | 99.6 | 99.8 |
| ‘C’ Selectivities | | | | |
| C₂₌ | 37.4 | 38.6 | 43.1 | 40.4 |
| C_{2≡} | 0.1 | 0.0 | 0.4 | 1.0 |
| C₂ | 42.0 | 35.7 | 24.0 | 14.4 |
| C₃₌ | 3.6 | 3.7 | 4.3 | 3.5 |
| C₃ | 1.9 | 1.7 | 1.4 | 0.1 |
| C₄₌ | 1.5 | 0.0 | 0.0 | 2.9 |
| C₂₊ | 86.4 | 79.8 | 73.3 | 62.4 |
| CO | 5.2 | 6.6 | 10.6 | 22.5 |
| CO₂ | 8.3 | 13.7 | 16.0 | 15.1 |
| H₂/CO molar ratio | 0.6 | 0.4 | 0.2 | 0.1 |

Higher per pass conversion of methane can reduce the capital cost of the process and so it is desirable to run at high methane conversion. However, the C₂₊ selectivity is lower when operated at lower feed CH₄/O₂ ratios, as evident from data in Table 1. As will be appreciated by one of skill in the art, and with the help of this disclosure, operating at lower feed CH₄/O₂ ratios requires the use of a diluent, usually steam, to control the exothermic nature of the OCM reaction. Use of a heavy diluent enhances the C₂₊ selectivity, thereby advantageously leading to a sum of methane conversion plus C₂₊ selectivity of greater than about 100%.

By choosing a heavy diluent like CO₂, a ratio of diffusivities of methane to oxygen can be enriched by about 10% and as such the local CH₄/O₂ molar ratio can be higher than the bulk (e.g., feed) CH₄/O₂ molar ratio. Further, the separation of CO₂ from the product mixture can also be economically done. Table 2 provides the molecular diffusivities of methane and oxygen in various diluents, as estimated by Chapman-Enskog equation.

TABLE 2

| | Diffusivities | | | | Ratio of diffusivities | |
|---|---|---|---|---|---|---|
| | In CO₂ | | In Water | | In water | In CO₂ |
| Temp, ° K | CH₄ | O₂ | CH₄ | O₂ | CH₄/O₂ | CH₄/O₂ |
| 873 | 1.13 | 1.00 | 1.61 | 1.59 | 1.01 | 1.13 |
| 973 | 1.35 | 1.20 | 1.96 | 1.92 | 1.02 | 1.13 |
| 1073 | 1.60 | 1.42 | 2.32 | 2.28 | 1.02 | 1.13 |
| 1173 | 1.86 | 1.64 | 2.72 | 2.66 | 1.02 | 1.13 |
| 1273 | 2.13 | 1.88 | 3.31 | 3.06 | 1.08 | 1.13 |

The data in Table 2 indicate that the use of CO₂ as heavy diluent in the feed has a positive effect to enhance local CH₄/O₂ ratio near the catalyst (e.g., at a catalyst surface), when compared to the use of water as a diluent. While an overall bulk feed CH₄/O₂ ratio determines the CH₄ conversion, the C₂₊ selectivity can be enhanced in the presence of a heavy diluent, thereby advantageously leading to a sum of methane conversion plus C₂+ selectivity of greater than about 100%.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed herein are provided solely for their disclosure prior to the filing date of the present application. Nothing herein is to be construed as an admission that the inventors are not entitled to antedate such disclosure by virtue of prior invention.

In any application before the United States Patent and Trademark Office, the Abstract of this application is provided for the purpose of satisfying the requirements of 37 C.F.R. §1.72 and the purpose stated in 37 C.F.R. §1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

The present disclosure is further illustrated by the following examples, which are not to be construed in any way as imposing limitations upon the scope thereof. On the contrary, it is to be clearly understood that resort can be had to various other aspects, embodiments, modifications, and equivalents thereof which, after reading the description herein, can be suggest to one of ordinary skill in the art without departing from the spirit of the present invention or the scope of the appended claims.

### ADDITIONAL DISCLOSURE

A first embodiment, which is a method for producing C₂ hydrocarbons comprising (a) introducing a reactant mixture to a reactor comprising a catalyst, wherein the reactant mixture comprises methane (CH₄), oxygen (O₂) and a heavy diluent, and wherein the reactant mixture is characterized by a bulk CH₄/O₂ molar ratio; (b) allowing at least a portion of the reactant mixture to contact a surface of the catalyst and react via an oxidative coupling of CH₄ reaction to form a product mixture, wherein the reactant mixture is characterized by a local CH₄/O₂ molar ratio on the catalyst surface, wherein the local CH₄/O₂ molar ratio is greater than the bulk CH₄/O₂ molar ratio, wherein the product mixture comprises C₂ hydrocarbons, and wherein a selectivity to C₂ hydrocarbons is increased by equal to or greater than about 1% when compared to a selectivity of an otherwise similar oxidative coupling of CH₄ reaction conducted with a similar bulk CH₄/O₂ molar ratio in the absence of the heavy diluent; and (c) recovering at least a portion of the product mixture from the reactor.

A second embodiment, which is the method of the first embodiment, wherein the heavy diluent comprises carbon dioxide (CO₂), silicon tetrafluoride (SiF₄), carbon tetrafluoride (CF₄), a heavy inert gas, argon (Ar), krypton (Kr), or combinations thereof.

A third embodiment, which is the method of any one of the first and the second embodiments, wherein the heavy diluent comprises CO₂.

A fourth embodiment, which is the method of any one of the first through the third embodiments, wherein the heavy diluent forms a heavy diluent-rich thin layer at the surface of the catalyst.

A fifth embodiment, which is the method of the fourth embodiment, wherein the CH₄ diffuses faster than O₂ through the heavy diluent-rich thin layer, at a given temperature.

A sixth embodiment, which is the method of any one of the first through the fifth embodiments, wherein the reactant mixture is characterized by a diffusivity ratio of CH₄/O₂ in a gas mixture comprising the heavy diluent that is increased by equal to or greater than about 5% when compared to a diffusivity ratio of CH₄/O₂ in an otherwise similar a gas mixture lacking the heavy diluent, at a given temperature.

A seventh embodiment, which is the method of any one of the first through the sixth embodiments, wherein the heavy diluent further comprises water, light inert gases, nitrogen, or combinations thereof.

An eighth embodiment, which is the method of any one of the first through the seventh embodiments, wherein the heavy diluent is characterized by a molecular weight of from about 35 g/mol to about 125 g/mol.

A ninth embodiment, which is the method of any one of the first through the eighth embodiments, wherein the heavy diluent is characterized by a thermal stability of equal to or less than about 1,200° C.

A tenth embodiment, which is the method of any one of the first through the ninth embodiments, wherein the heavy diluent is present in the reactant mixture in an amount of from about 10 mol % to about 80 mol %.

An eleventh embodiment, which is the method of any one of the first through the tenth embodiments, wherein the bulk CH₄/O₂ molar ratio is from about 1:1 to about 20:1.

A twelfth embodiment, which is the method of any one of the first through the eleventh embodiments, wherein the reactor is characterized by a residence time of from about 1 millisecond to about 2 seconds.

A thirteenth embodiment, which is the method of any one of the first through the twelfth embodiments, wherein the reactor is characterized by a pressure of from about ambient pressure to about 500 psig.

A fourteenth embodiment, which is the method of any one of the first through the thirteenth embodiments, wherein the reactor comprises an adiabatic reactor, an autothermal reactor, a tubular reactor, a cooled tubular reactor, a continuous flow reactor, a fixed bed reactor, a fluidized bed reactor, or combinations thereof.

A fifteenth embodiment, which is the method of any one of the first through the fourteenth embodiments, wherein the reactor is characterized by a weight hourly space velocity of from about 1,000 h to about 1,000,000 h⁻¹.

A sixteenth embodiment, which is the method of any one of the first through the fifteenth embodiments, wherein the catalyst catalyzes the oxidative coupling of CH₄ reaction.

A seventeenth embodiment, which is the method of any one of the first through the sixteenth embodiments, wherein the catalyst comprises basic oxides; mixtures of basic oxides; redox elements; redox elements with basic properties; mixtures of redox elements with basic properties; mixtures of redox elements with basic properties promoted with alkali and/or alkaline earth metals; rare earth metal oxides; mixtures of rare earth metal oxides; mixtures of rare earth metal oxides promoted by alkali and/or alkaline earth metals; manganese; manganese compounds; lanthanum; lanthanum compounds; sodium; sodium compounds; cesium; cesium compounds; calcium; calcium compounds; or combinations thereof.

An eighteenth embodiment, which is the method of any one of the first through the seventeenth embodiments, wherein the catalyst comprises CaO, MgO, BaO, CaO—MgO, CaO—BaO, Li/MgO, MnO₂, W₂O₃, SnO₂, MnO₂—W₂O₃, MnO₂—W₂O₃—Na₂O, MnO₂—W₂O₃—Li₂O, La₂O₃, SrO/La₂O₃, CeO₂, Ce₂O₃, La/MgO, La₂O₃—CeO₂, La₂O₃—CeO₂—Na₂O, La₂O₃—CeO₂—CaO, Na—Mn—La₂O₃/Al₂O₃, Na—Mn—O/SiO₂, Na₂WO₄—Mn/SiO₂, Na₂WO₄—Mn—O/SiO₂, or combinations thereof.

A nineteenth embodiment, which is the method of any one of the first through the eighteenth embodiments further excluding CO₂ reforming of CH₄.

A twentieth embodiment, which is the method of any one of the first through the nineteenth embodiments, wherein the catalyst does not catalyze CO₂ reforming of CH₄.

A twenty-first embodiment, which is the method of any one of the first through the twentieth embodiments, wherein the catalyst excludes nickel, a noble metal, rhodium, ruthenium, platinum, palladium, or combinations thereof.

A twenty-second embodiment, which is the method of any one of the first through the twenty-first embodiments, wherein a methane conversion is from about 5% to about 50%.

A twenty-third embodiment, which is the method of any one of the first through the twenty-second embodiments, wherein the C₂ hydrocarbons comprise ethylene and ethane.

A twenty-fourth embodiment, which is the method of any one of the first through the twenty-third embodiments, wherein the selectivity to C₂ hydrocarbons is from about 50% to about 90%.

A twenty-fifth embodiment, which is the method of any one of the first through the twenty-fourth embodiments, wherein a selectivity to ethylene is from about 30% to about 50%.

A twenty-sixth embodiment, which is the method of any one of the first through the twenty-fifth embodiments, wherein the product mixture comprises C₂₊ hydrocarbons, wherein the C₂₊ hydrocarbons comprise C₂ hydrocarbons and C₃ hydrocarbons.

A twenty-seventh embodiment, which is the method of the twenty-sixth embodiment, wherein the C₃ hydrocarbons comprise propylene and propane.

A twenty-eighth embodiment, which is the method of any one of the first through the twenty-seventh embodiments, wherein the C₂₊ hydrocarbons further comprise C₄ hydrocarbons.

A twenty-ninth embodiment, which is the method of any one of the first through the twenty-eighth embodiments, wherein a selectivity to C₂₊ hydrocarbons is from about 55% to about 95%.

A thirtieth embodiment, which is the method of any one of the first through the twenty-ninth embodiments, wherein equal to or greater than about 5 mol % of the reactant mixture is converted to ethylene.

A thirty-first embodiment, which is the method of any one of the first through the thirtieth embodiments, wherein equal to or greater than about 10 mol % of the reactant mixture is converted to C₂ hydrocarbons.

A thirty-second embodiment, which is the method of any one of the first through the thirty-first embodiments, wherein equal to or greater than about 12 mol % of the reactant mixture is converted to C₂₊ hydrocarbons.

A thirty-third embodiment, which is the method of any one of the first through the thirty-second embodiments, wherein the product mixture further comprises at least a portion of the heavy diluent and unreacted methane.

A thirty-fourth embodiment, which is the method of the thirty-third embodiment, wherein at least a portion of the heavy diluent is separated from the product mixture to yield a recovered heavy diluent.

A thirty-fifth embodiment, which is the method of the thirty-fourth embodiment, wherein at least a portion of the recovered heavy diluent is recycled to the reactant mixture.

A thirty-sixth embodiment, which is the method of any one of the first through the thirty-fifth embodiments, wherein at least a portion of the C₂₊ hydrocarbons is separated from the product mixture to yield recovered C₂₊ hydrocarbons.

A thirty-seventh embodiment, which is the method of the thirty-sixth embodiment, wherein at least a portion of the recovered C₂₊ hydrocarbons is used for ethylene production.

A thirty-eighth embodiment, which is the method of any one of the first through the thirty-seventh embodiments, wherein a at least a portion of the unreacted methane is separated from the product mixture to yield recovered methane.

A thirty-ninth embodiment, which is the method of the thirty-eighth embodiment, wherein at least a portion of the recovered methane is recycled to the reactant mixture.

A fortieth embodiment, which is the method of any one of the first through the thirty-ninth embodiments, wherein the product mixture further comprises synthesis gas.

A forty-first embodiment, which is the method of the fortieth embodiment, wherein at least a portion of the unreacted methane and at least a portion of the synthesis gas are separated from the product mixture to yield a fuel gas mixture.

A forty-second embodiment, which is the method of the forty-first embodiment, wherein at least a portion of the fuel gas mixture is used as a source of fuel for generating energy.

A forty-third embodiment, which is a method for producing ethylene comprising (a) introducing a reactant mixture to a reactor comprising a catalyst, wherein the reactant mixture comprises methane (CH₄), oxygen (O₂) and carbon dioxide (CO₂), and wherein the reactant mixture is characterized by a bulk CH₄/O₂ molar ratio of from about 4:1 to about 8:1; (b) allowing at least a portion of the reactant mixture to contact a surface of the catalyst and react via an oxidative coupling of CH₄ reaction to form a product mixture, wherein the reactant mixture is characterized by a local CH₄/O₂ molar ratio on the catalyst surface, wherein the local CH₄/O₂ molar ratio is greater than the bulk CH₄/O₂ molar ratio, wherein the product mixture comprises C₂ hydrocarbons, and wherein a selectivity to C₂ hydrocarbons is increased by equal to or greater than about 5% when compared to a selectivity of an otherwise similar oxidative coupling of CH₄ reaction conducted with a similar reactant mixture lacking CO₂; (c) recovering at least a portion of the product mixture from the reactor; (d) separating at least a portion of C₂₊ hydrocarbons from the product mixture to yield recovered C₂₊ hydrocarbons; and (e) using at least a portion of the recovered C₂₊ hydrocarbons to produce ethylene.

A forty-fourth embodiment, which is the method of the forty-third embodiment, wherein (e) using at least a portion of the recovered C₂₊ hydrocarbons to produce ethylene comprises separating ethylene from the C₂₊ hydrocarbons to yield recovered ethylene.

A forty-fifth embodiment, which is the method of any one of the forty-third and the forty-fourth embodiments, wherein (e) using at least a portion of the recovered C₂₊ hydrocarbons to produce ethylene comprises converting C₂₊ hydrocarbons to ethylene.

A forty-sixth embodiment, which is the method of any one of the forty-third through the forty-fifth embodiments further excluding CO₂ reforming of CH₄.

While embodiments of the disclosure have been shown and described, modifications thereof can be made without departing from the spirit and teachings of the invention. The embodiments and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

What is claimed is:

1. A method for producing C₂ hydrocarbons comprising:
(a) introducing a reactant mixture to a reactor comprising a catalyst, wherein the reactant mixture comprises methane (CH₄), oxygen (O₂) and a heavy diluent, and wherein the reactant mixture is characterized by a bulk CH₄/O₂ molar ratio;
(b) allowing at least a portion of the reactant mixture to contact a surface of the catalyst and react via an oxidative coupling of CH₄ reaction to form a product mixture, wherein the reactant mixture is characterized by a local CH₄/O₂ molar ratio on the catalyst surface, wherein the local CH₄/O₂ molar ratio is greater than the bulk CH₄/O₂ molar ratio, wherein the product mixture comprises C₂ hydrocarbons, and wherein a selectivity to C₂ hydrocarbons is increased by equal to or greater than about 1% when compared to a selectivity of an otherwise similar oxidative coupling of CH₄ reaction conducted with a similar bulk CH₄/O₂ molar ratio in the absence of the heavy diluent; and
(c) recovering at least a portion of the product mixture from the reactor.

2. The method of claim 1, wherein the heavy diluent comprises carbon dioxide (CO₂), silicon tetrafluoride (SiF₄), carbon tetrafluoride (CF₄), a heavy inert gas, argon (Ar), krypton (Kr), or combinations thereof.

3. The method of claim 1, wherein the heavy diluent comprises CO₂.

4. The method of claim 1, wherein the heavy diluent forms a heavy diluent-rich thin layer at the surface of the catalyst.

5. The method of claim 4, wherein the CH₄ diffuses faster than O₂ through the heavy diluent-rich thin layer, at a given temperature.

6. The method of claim 1, wherein the reactant mixture is characterized by a diffusivity ratio of CH₄/O₂ in a gas mixture comprising the heavy diluent that is increased by equal to or greater than about 5% when compared to a diffusivity ratio of CH₄/O₂ in an otherwise similar a gas mixture lacking the heavy diluent, at a given temperature.

7. The method of claim 1, wherein the heavy diluent further comprises water, light inert gases, nitrogen, or combinations thereof.

8. The method of claim 1, wherein the heavy diluent is characterized by a molecular weight of from about 35 g/mol to about 125 g/mol.

9. The method of claim 1, wherein the heavy diluent is characterized by a thermal stability of equal to or less than about 1,200° C.

10. The method of claim 1, wherein the heavy diluent is present in the reactant mixture in an amount of from about 10 mol % to about 80 mol %.

11. The method of claim 1 further excluding CO₂ reforming of CH₄.

12. The method of claim 1, wherein a methane conversion is from about 5% to about 50%.

13. The method of claim 1, wherein the selectivity to C₂ hydrocarbons is from about 50% to about 90%.

14. The method of claim 1, wherein a selectivity to ethylene is from about 30% to about 50%.

15. The method of claim 1, wherein the product mixture comprises C₂₊ hydrocarbons and wherein a selectivity to C₂₊ hydrocarbons is from about 55% to about 95%.

16. The method of claim 1, wherein equal to or greater than about 5 mol % of the reactant mixture is converted to ethylene, wherein equal to or greater than about 10 mol % of the reactant mixture is converted to C₂ hydrocarbons, and wherein equal to or greater than about 12 mol % of the reactant mixture is converted to C₂₊ hydrocarbons.

17. The method of claim 1, wherein the product mixture further comprises at least a portion of the heavy diluent and unreacted methane, wherein at least a portion of the heavy diluent is separated from the product mixture to yield a recovered heavy diluent, wherein at least a portion of the recovered heavy diluent is recycled to the reactant mixture, wherein at least a portion of the unreacted methane is separated from the product mixture to yield recovered methane, and wherein at least a portion of the recovered methane is recycled to the reactant mixture.

18. The method of claim 1, wherein at least a portion of the C₂₊ hydrocarbons is separated from the product mixture to yield recovered C₂₊ hydrocarbons and wherein at least a portion of the recovered C₂₊ hydrocarbons is used for ethylene production.

19. The method of claim 17, wherein the product mixture further comprises synthesis gas and wherein at least a portion of the unreacted methane and at least a portion of the synthesis gas are separated from the product mixture to yield a fuel gas mixture.

20. A method for producing ethylene comprising:
(a) introducing a reactant mixture to a reactor comprising a catalyst, wherein the reactant mixture comprises methane (CH₄), oxygen (O₂) and carbon dioxide (CO₂), and wherein the reactant mixture is characterized by a bulk CH₄/O₂ molar ratio of from about 4:1 to about 8:1;
(b) allowing at least a portion of the reactant mixture to contact a surface of the catalyst and react via an oxidative coupling of CH₄ reaction to form a product mixture, wherein the reactant mixture is characterized by a local CH₄/O₂ molar ratio on the catalyst surface, wherein the local CH₄/O₂ molar ratio is greater than the bulk CH₄/O₂ molar ratio, wherein the product mixture comprises C₂ hydrocarbons, and wherein a selectivity to C₂ hydrocarbons is increased by equal to or greater than about 5% when compared to a selectivity of an otherwise similar oxidative coupling of CH₄ reaction conducted with a similar reactant mixture lacking CO₂;
(c) recovering at least a portion of the product mixture from the reactor;
(d) separating at least a portion of C₂₊ hydrocarbons from the product mixture to yield recovered C₂₊ hydrocarbons; and
(e) using at least a portion of the recovered C₂₊ hydrocarbons to produce ethylene.
